(19) United States
(12) Patent Application Publication
(10) Pub. No.: US 2019/0091738 A1
(43) Pub. Date: Mar. 28, 2019

(54) CLEANING SYSTEM FOR A VEHICLE

(71) Applicant: Ford Global Technologies, LLC, Dearborn, MI (US)

(72) Inventor: Jay Chen, Sylvania, OH (US)

(73) Assignee: Ford Global Technologies, LLC, Dearborn, MI (US)

(21) Appl. No.: 15/713,146

(22) Filed: Sep. 22, 2017

(51) Int. Cl. B08B 7/00; G01J 1/42; A61L 2/10; B60S 1/64

(52) U.S. Cl. CPC B08B 7/0057; B60S 1/64; A61L 2/10; G01J 1/429

(57) ABSTRACT

A cleaning system for a vehicle is described and methods of using the system. The method may include: at a vehicle computer: receiving data from at least one environmental sensor; based on the data, determining an ultraviolet (UV) dosage for an interior surface of a cabin; and based on the determination, controlling UV light according to the dosage.

### BACKGROUND

Ultraviolet light (such as UV-B and UV-C) may be used as a germicidal agent. Disinfection may be achieved by killing or inactivating various microorganisms.

### BRIEF DESCRIPTION OF THE DRAWINGS

FIG. 1 is a perspective view of a cleaning system for a cabin of a vehicle.

FIG. 2 is a schematic view of the cleaning system of the vehicle shown in FIG. 1.

FIG. 3 is a schematic view of a light source and a light detector of the cleaning system.

FIG. 4 is a flow diagram illustrating a vehicle interior cleaning process executable by a computer of the cleaning system.

FIG. 5 is a graphical depiction of several bands of ultraviolet (UV) wavelength, each associated with a different type of UV light element.

### DETAILED DESCRIPTION

A cleaning system is described and a method of using the system. According to one illustrative example, the method includes: at a vehicle computer: receiving data from at least one environmental sensor; based on the data, determining an ultraviolet (UV) dosage for an interior surface of a cabin; and based on the determination, controlling UV light according to the dosage.

According to the at least one example set forth above, controlling further comprises actuating a light source within the cabin which directs the light toward the surface.

According to the at least one example set forth above, the light comprises a band within 240-280 nanometers.

According to the at least one example set forth above, the dosage is based on a light intensity, an exposure duration, and at least one function based on the data.

According to the at least one example set forth above, the method further includes de-actuating a light source at an expiration of the duration.

According to the at least one example set forth above, the method further includes adjusting at least one climate control parameter based on the determination.

According to the at least one example set forth above, controlling further comprises changing an intensity of the light based on feedback from a detector at the surface.

According to the at least one example set forth above, the determination further comprises increasing an intensity of the light based on a relative humidity being greater than a threshold.

According to the at least one example set forth above, the determination further comprises decreasing an intensity of the light based on a relative temperature being less than a first threshold or greater than a second threshold.

According to the at least one example set forth above, the determination further comprises increasing an intensity of the light based on moisture at the surface being greater than a threshold.

According to the at least one example set forth above, the determination further comprises decreasing an intensity of the light based on a measurement of UV sunlight at the surface.

According to the at least one example set forth above, the method further includes inhibiting UV light emission based on an occupied state of the vehicle or a user ingres sing.

According to the at least one example set forth above, the method further includes inhibiting UV light emission based on relative airflow being greater than a threshold.

According to the at least one example set forth above, the method further includes inhibiting UV light emission based an open state of vehicle windows.

According to the at least one example set forth above, the dosage is based on a selected sterilization level.

According to another illustrative example, a system is disclosed. The system may include a computer, comprising processor and memory storing instructions executable by the processor, the instructions comprising, to: receive data from at least one environmental sensor; based on the data, determine an ultraviolet (UV) dosage for an interior surface of a cabin; and based on the determination, control UV light according to the dosage.

According to the at least one example set forth above, the system also may include: a lighting system coupled to the computer.

According to the at least one example set forth above, the lighting system comprises a light source and a detector which provides UV light intensity feedback.

According to the at least one example set forth above, the instructions further comprise, to: determine the dosage based on one of a relative humidity, a relative temperature, or a moisture at the surface.

According to the at least one example set forth above, the instructions further comprise, to: adjust the dosage based on a measurement of UV sunlight at the surface.

According to the at least one example, a computer is disclosed that is programmed to execute any combination of the examples set forth above.

According to the at least one example, a computer is disclosed that is programmed to execute any combination of the examples of the method(s) set forth above.

According to the at least one example, a computer program product is disclosed that includes a computer readable medium storing instructions executable by a computer processor, wherein the instructions include any combination of the instruction examples set forth above.

According to the at least one example, a computer program product is disclosed that includes a computer readable medium that stores instructions executable by a computer processor, wherein the instructions include any combination of the examples of the method(s) set forth above.

Now turning to the figures, wherein like numerals indicate like parts throughout the several views, there is shown a cleaning system 10 for a vehicle 12 that includes a computer 14, a lighting system 16 (e.g., providing ultraviolet (UV) light), and one or more environmental sensors 18. As will be described in detail below, the computer 14 may receive sensor data from sensor(s) 18, determine a light emission dosage, and, when a cabin 20 of the vehicle 12 contains no users (e.g., human passengers or occupants), the computer 14 may control the lighting system 16 to emit light to impinge upon and clean an interior surface 22 of the cabin 20 (e.g., it may actuate the lighting system 16 to an ON state). More particularly, computer 14 may tailor a dosage (e.g., UV-C light exposure for a period of application time) to target one or more types of surface contaminants (e.g., bacteria, viruses, fungi, other pathogens, etc.). For example, using the environmental sensor data collected via sensors 18, computer 14 in some instances may identify potential contaminants and, based on that identification, determine an appropriate dosage. Non-limiting examples of environmental sensor data include cabin humidity data, cabin temperature data, cabin surface sunlight data, surface moisture data, cabin airflow data, and the like. According to one example, in determining the dosage, computer 14 may determine a wavelength (or band), determine an intensity (of UV light), and/or determine an exposure duration (of the UV light) which may be suitable for eliminating, exterminating, or killing the identified contaminant while avoiding overdosing the surface 22—as overdosing may result in deterioration of vehicle interior components (e.g., such as interior trim, moldings, etc. which may comprise rubber, plastic, or other materials which degrade in the presence of UV light). In addition, the computer 14 may actuate lighting system 16 to an OFF state following an expiration of the exposure duration or when, during the duration, the computer 14 determines that a user may be entering the vehicle 12. In at least some instances, in order to inhibit or minimize the likelihood of contaminant growth within the cabin 20, the computer 14 may control at least one climate control parameter following the UV dosage application (e.g., controlling cabin humidity, temperature, airflow, etc.). Computer 14 and other aspects of the cleaning system 10 are described in greater detail below.

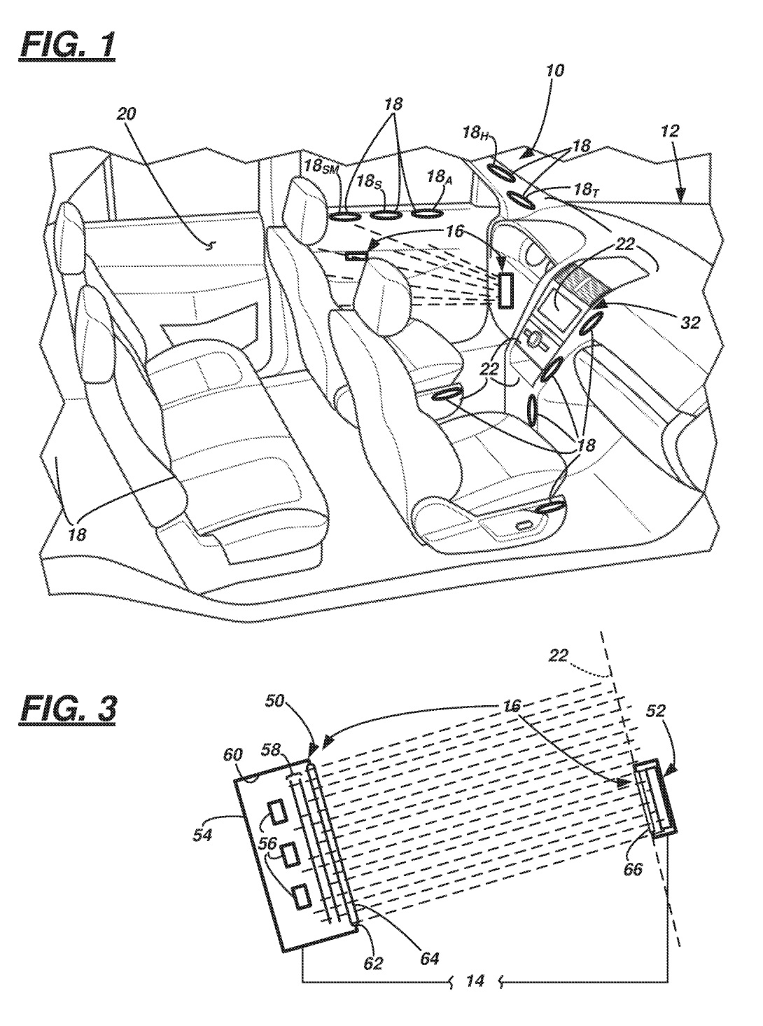

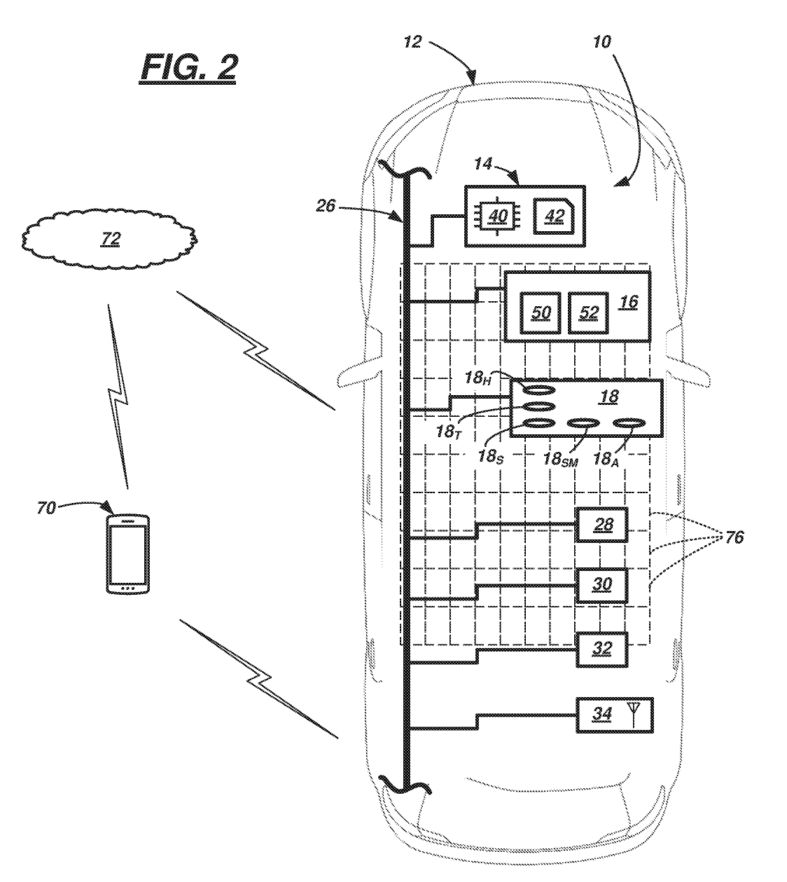

FIGS. 1-2 illustrate an illustrative vehicle 12 that may comprise cleaning system 10. Vehicle 12 is shown as a passenger car; however, vehicle 12 could also be a truck, sports utility vehicle (SUV), recreational vehicle, bus, train car, aircraft, or the like that includes the cleaning system 10. Vehicle 12 may be operated in any one of a number of autonomous modes. In at least one example, vehicle 12 may operate as an autonomous taxi, autonomous school bus, or the like e.g., operating in a fully autonomous mode (e.g., a level 5), as defined by the Society of Automotive Engineers (SAE) (which has defined operation at levels 0-5). For example, at levels 0-2, a human driver monitors or controls the majority of the driving tasks, often with no help from the vehicle 12. For example, at level 0 (“no automation”), a human driver is responsible for all vehicle operations. At level 1 (“driver assistance”), the vehicle 12 sometimes assists with steering, acceleration, or braking, but the driver is still responsible for the vast majority of the vehicle control. At level 2 (“partial automation”), the vehicle 12 can control steering, acceleration, and braking under certain circumstances without human interaction. At levels 3-5, the vehicle 12 assumes more driving-related tasks. At level 3 (“conditional automation”), the vehicle 12 can handle steering, acceleration, and braking under certain circumstances, as well as monitoring of the driving environment. Level 3 may require the driver to intervene occasionally, however. At level 4 (“high automation”), the vehicle 12 can handle the same tasks as at level 3 but without relying on the driver to intervene in certain driving modes. At level 5 (“full automation”), the vehicle 12 can handle all tasks without any driver intervention. In at least one example, vehicle 12 includes one or more autonomous driving systems, one or more autonomous driving computers, and the like to enable operation at level 5 and thus may operate in a fully autonomous mode. In this fully autonomous mode, the vehicle 12 may operate as an autonomous taxi or the like.

When vehicle 12 is used as an autonomous taxi, bus, or the like, the cabin 20 typically will be used daily by a number of different users. As used herein, a cabin is a region of vehicle 12 adapted with passenger seating. The cleaning system 10 may facilitate some cleaning between at least some occupancies of these different users—e.g., during periods of less frequent vehicle use. The cleaning system 10 described herein may be particularly desirable in relatively small, enclosed environments: since smaller and enclosed regions often have temperatures suitable for human comfort (e.g., such as cabin 20) but which also can harbor and/or promote the growth of infectious pathogens such as bacteria, viruses, and fungi; and since both infected and un-infected users regularly may utilize vehicle 12—e.g., thus making the vehicle cabin 20, without the cleaning system 10, a more likely vessel for spreading infection. Thus, according to at least one example, cleaning system 10 may be used to minimize the spread of respiratory, gastrointestinal, and other illnesses (e.g., which can be spread by user contact with contaminated surfaces 22 within the vehicle 12).

According to one example, cleaning system 10 comprises a wired or wireless communication network connection 26 which facilitates communication between one or more of: computer 14, lighting system 16, environmental sensor(s) 18, an occupant detection system 28, a climate control system 30, a human-machine interface (HMI) module 32, and a telematics module 34. In at least one example, the connection 26 includes one or more of a controller area network (CAN) bus, Ethernet, Local Interconnect Network (LIN), a fiber optic connection, or the like. Other examples also exist. For example, alternatively or in combination with e.g., a CAN bus, connection 26 could comprise one or more discrete wired or wireless connections (e.g., between the sensors 18 and computer 14, between the lighting system 16 and computer 14, etc.).

Computer 14 may be a single computer (or multiple computing devices—e.g., shared with other vehicle systems and/or subsystems). Computer 14 may be a body control module (BCM); however, this is merely one non-limiting example. Computer 14 may comprise a processor 40 (e.g., or processing circuit) coupled to memory 42. For example, processor 40 can be any type of device capable of processing electronic instructions, non-limiting examples including a microprocessor, a microcontroller or controller, an application specific integrated circuit (ASIC), etc.—just to name a few. In general, computer 14 may be programmed to execute digitally-stored instructions, which may be stored in memory 42, which enable the computer 14, among other things, to: receive sensor data from at least one environmental sensor 18; determine at least one light source parameter based on the sensor data; determine a contaminant type based on the sensor data; actuate lighting system 16 based on the determined parameter and/or based on the contaminant type; execute a combination of these exemplary instructions; or the like. Other programmable instructions executable by processor 40 will be discussed in greater detail below.

Memory 42 may include any non-transitory computer usable or readable medium, which may include one or more storage devices or articles. Exemplary non-transitory computer usable storage devices include conventional hard disk, solid-state memory, random access memory (RAM), read-only memory (ROM), erasable programmable read-only memory (EPROM), electrically erasable programmable read-only memory (EEPROM), as well as any other volatile or non-volatile media. Non-volatile media include, for example, optical or magnetic disks and other persistent memory, and volatile media, for example, also may include dynamic random access memory (DRAM). These storage devices are non-limiting examples; e.g., other forms of computer-readable media exist and include magnetic media, compact disc ROM (CD-ROMs), digital video disc (DVDs), other optical media, any suitable memory chip or cartridge, or any other medium from which a computer can read. As discussed above, memory 42 may store one or more computer program products which may be embodied as software, firmware, or other programming instructions executable by the processor 40.

Lighting system 16 (also illustrated in FIG. 3) may comprise any suitable light emitting device which cleans a cabin interior surface 22 when directed thereat. As used herein, an interior surface is an interior surface of vehicle cabin 22 and should be broadly construed to include any surfaces within the vehicle 12—including but not limited to any suitable surface of a vehicle instrument panel, any suitable touchscreen surface of the vehicle 12, any suitable surface of vehicle seating, an interior surface of a vehicle door or vehicle door panel, a vehicle steering wheel (if available), an interior surface of a vehicle window or window pane, or the like.

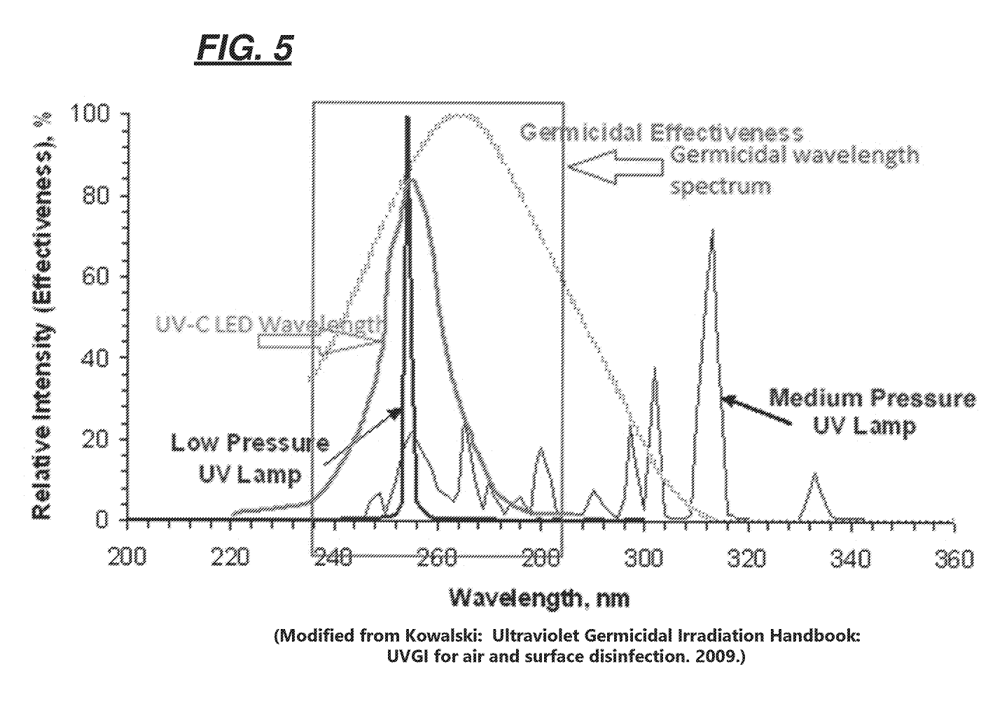

According to one example, the lighting system 16 comprises a light source 50 and a light detector 52. Light source 50 may comprise a housing 54, one or more light elements 56, and one or more filters 58. Non-limiting examples of light elements 56 include UV-emitting gas discharge lamps, UV-emitting fluorescent bulbs, UV-emitting light emitting diodes (LEDs), UV-emitting organic light emitting diodes (OLEDs), and the like. According to at least one example, elements 56 comprise one or more UV-emitting gas discharge lamp(s)—e.g., as these types of elements may have suitable sterilization parameters (e.g., a narrow bandwidth of 4 nanometers (nm), typically centered at about 253.7 nm which is a suitable center frequency for killing bacteria, viruses, fungi, and other contaminants). As shown in FIG. 5, UV-LEDs and UV-OLEDs and fluorescent bulbs may have bandwidths greater than 20 nm, require broader filtering, etc.

FIG. 3 illustrates several light elements 56 (e.g., LEDs) carried within a cavity 60 of the housing 54; more or fewer elements 56 may be used in other examples. In this example, the filters 58 are positioned between the elements 56 and an opening 62 of the housing 54 so that emitted light from elements 56 is filtered. The filters 58 may block transmission of any suitable wavelengths of light from exiting housing 54. The term filter should be construed broadly to include a film, screen, or other layer of light blocking material—e.g., which in some instances, include dyes and/or other polarizing apparatuses. In at least one example, the light source 50 comprises a lens and/or diffuser 64—e.g., positioned within the opening 62 to control the directionality, focus, etc. of the emitted light.

According to one example, light source 50 may emit ultraviolet (UV) light having a peak wavelength within a band of 240-280 nanometers (nm); and according to at least one example, the UV center frequency is 253.7 nm (e.g., having a +/−2 nm bandwidth), as discussed above. As will be described below, computer 14 may control the emitted wavelength, intensity, and/or an exposure duration. For example, to control the wavelength, computer 14 may selectively actuate or move one or more filters 58 (e.g., mechanically positioning them (and/or displacing from) between the elements 56 and opening 62). In another example of wavelength control, computer 14 may selectively actuate one or more of elements 56, wherein at least one of the elements 56 emits light at a different wavelength than the others. These and other techniques may be used singly or in combination with one another (by computer 14) to control wavelength output.

According to one example, the peak wavelength is selected to be between 240-280 nm because this light region of the spectrum may photo-catalyze the formation of deoxyribonucleic acid (DNA) pyrimidine dimers—which damages cellular DNA and triggers cell death (e.g., in a pathogen). Also, the use of filter(s) 58 (e.g., such as a high pass filter to inhibit light wavelengths less than 200 nm) desirably may minimize the formation of ozone (O₃) within cabin 20.

With respect to controlling light emission intensity, computer 14 may control intensity by selectively controlling a voltage or current to at least some of the elements 56—e.g., in instances where greater current or voltage results in a higher intensity output from the elements 56. In other examples, which may be used alternatively or in combination herewith, computer 14 selectively may actuate elements 56. For instance, in the illustrated example (FIG. 3), computer 14 could minimize intensity by illuminating only one or two of the elements 56. And to increase intensity, computer 14 could actuate all three elements 56. Like the discussion of wavelength control, this also is merely an example. Other examples exist.

As will be discussed below, computer-selected intensity from light source 50 (e.g., as well as light emission duration) may depend on the targeted contaminant. For example, computer 14 may determine a likely contaminant based on sensor data from the environmental sensors 18, and based on this determination, computer 14 may control the intensity—which intensities may differ for each of bacteria, fungi, bacterial endospores, etc. Also, computer 14 may adjust the intensity based on a desired cellular logarithmic kill ratio or so-called sterilization level (e.g., 90%, 99%, 99.99%, etc.)—e.g., higher levels having a greater likelihood of killing the contaminant. These will be discussed more below.

In at least one example, multiple light sources 50 may be positioned within vehicle cabin 20, and at least some of these sources 50 may have different arrangements or quantities of light elements 56. Further, multiple light sources 50 may correspond to a single detector 52, or for example, each light source 50 may have a corresponding detector 52.

Further, while FIG. 1 illustrates a light source 50 in an instrument panel, in other examples, a light source 50 may be located in a vehicle headliner, in a vehicle A-, B-, etc. pillar, in a vehicle center console, in a vehicle door panel, or the like. Intensity of each respective light source 50 may be controlled at least partially based on a relative distance between the source 50 and a targeted surface 22 (e.g., accounting for intensity received at the surface 22 being inversely proportional to the square of the distance between the source 50 and the targeted surface 22). Other aspects and techniques for using the light source(s) 50 will be discussed below.

Light detector 52 may be any suitable electronic device for detecting and measuring an intensity of impinging light (e.g., power per unit area) at an interior surface 22. For example, the detector 52 may be located at, on, or even at least partially within the interior surface 22. According to one non-limiting example (FIG. 3), detector 52 comprises a UV-sensitive window 66 which may be approximately flush with surface 22 (i.e., more particularly, window 66 may be an element which is electrically responsive to UV light). Using window 66, detector 52 may provide as output an electrical signal, wherein the magnitude of the current or voltage of the signal may correspond with a magnitude of intensity impinging upon the window 66.

According to at least one example, the light source 50 and/or detector 52 may be positioned and oriented (relative to one another) so that window 66 receives incident light from source 50 (e.g., having an angle of incidence of approximately 0° (e.g., between +/−5° of normal); however, this is not required in all examples. As will be appreciated from the discussion below, smaller angles of incidence may result in light source 50 using less power in cleaning applications, as less UV light may be reflected from window 66 (and in some degree, surface 22). Further, in some examples, if the angle of incidence is too great, all or most of the UV light may be reflected at the window 66—e.g., resulting in the detector 52 being unable to measure UV light intensity from the source 50.

According to one example, detector 42 may detect UV intensity between 0 and 2500 microWatts/centimeter̂2 (μW/cm²). As will be described more below, light source 50 may be configured to direct light at the surface 22 (and consequently at window 66) having an intensity between 500-2000 μW/cm². Light detector 52 may be coupled to computer 14 (and/or light source 50) in order to provide feedback data regarding how much UV light is actually being received at the surface 22. Thus, for example, computer 14 may instruct light source 50 to emit light having a predetermined intensity, detector 52 may measure the UV light intensity at surface 22, detector 52 may provide a measurement via a feedback loop to computer 14 (and/or to light source 50), and, based on the measurement, light source 50 may increase or decrease its power output in accordance with the instruction from computer 14. Further, based on the intensity of UV light received via the detector 52, computer 14 may maintain the controlled application of UV light for an exposure duration so that a predetermined dosage (e.g., energy per unit area) may be received at surface 22. Other aspects and examples of UV dosages will be described more below.

As discussed above, the cleaning system 10 may comprise one or more environmental sensors 18. And in at least one example, the system 10 comprises a plurality of sensors 18. At least some of these sensors 18 may be configured to receive different types of sensor data. For example, according to one example, system 10 comprises: a cabin humidity sensor 18_{H}, a cabin temperature sensor 18_{T}, a surface moisture sensor 18_{SM}, a sunlight sensor 18_{S}, an airflow sensor 18_{A}, or a combination thereof. Each of the sensors 18 may provide an electrical output to computer 14 which may be correlated thereby to an analog value (e.g., an analog temperature, an analog humidity, an analog moisture level, an analog UV sunlight level, an analog airflow level, etc.) and/or be compared with predetermined threshold values (e.g., which may be associated with contaminant growth, activity, etc.).

In FIG. 1, locations of these and other sensors 18 are shown for purposes of illustration only (i.e., and are not intended to be limiting). For example, the system 10 may comprise more than one humidity sensor 18_{H} and the plurality of humidity sensors 18_{H} could be located on or near a vehicle A-pillar (as shown) and/or elsewhere in the cabin 20. The same may be true for cabin temperature sensors 18_{T}, surface moisture sensors 18_{SM}, sunlight sensors 18_{S}, airflow sensors 18_{A}, etc. (i.e., there may be more than one of each and the respective locations may differ in other examples).

Turning now to the occupant detection system 28, system 28 may comprise any number of electronic devices which may be at least partially within the cabin 20 and which are coupled to computer 14 and which thereby enable the computer 14 to determine whether a user (or animal, pet, etc.) is within the vehicle 12. Non-limiting examples of such electronic devices include motion detection sensors within cabin 20, infrared or thermal sensors within cabin 20, pressure and/or proximity sensors (e.g., within vehicle seating, arm rests, etc.), imaging sensors within cabin 20 (e.g., such as complementary metal oxide semiconductor (CMOS) and charge-coupled device (CCD) cameras), and the like. The system 28 may comprise other cabin occupancy detection devices as well—e.g., including wireless signal (e.g., Bluetooth), door ajar signals, vehicle pitch and roll sensors, etc.). Thus, system 28 may comprise a computer processing device (not shown) which includes instructions enabling it to detect vehicle occupancy based on one or more inputs of the exemplary electronic devices discussed above. Furthermore, this computer processing device of system 28 may be programmed to increment a counter for vehicle occupants ingressing vehicle 12 and decrement the counter for occupants egressing the vehicle 12. (In other examples, these or similar programming instructions may be stored in memory 42 and executed using processor 40 of computer 14). Thus, in at least one example, occupant detection system 28 may determine an occupancy status and report this status to computer 14, as will be described in greater detail below.

Climate control system 30 may comprise any suitable components for providing cabin comfort by controlling temperature, humidity, airflow, etc. For example, system 30 may include a heating unit, an air-conditioning (AC) unit, a humidifier, a blower or fan, a manifold and plurality of air ducts opening into cabin 20—none of which are shown in the figures. Typically, climate control system 30 includes a computer processing device (e.g., having a processor and memory) which controls the heating and AC units, the blower, the humidifier, etc. In some instances, these components are programmable by the user or a vehicle or original equipment manufacturer. Further, in at least one example, the computer processing device may be adapted to receive (e.g., via network 26) an instruction from computer 14 requesting it to adjust a climate control parameter. For example, as explained more below, controlling the parameter may include selectively (and respectively) increasing or decreasing one or more of vehicle cabin temperature, cabin humidity, or forced air pressure (e.g., airflow) within the cabin 20.

Human-machine interface (HMI) module 32—also shown in FIG. 2—may include any suitable input and/or output devices such as switches, knobs, controls, etc.—e.g., on a vehicle instrument panel, steering wheel, etc. of vehicle 12—which are coupled communicatively to computer 14. In one non-limiting example, HMI module 32 may comprise an interactive touch screen or display which provides navigation information (e.g., including text, images, etc.) to the vehicle user and permits the user to enter a desired destination for the vehicle 12 in a fully autonomous mode to transport the user. Such interactive devices further may be used to notify the user that the vehicle interior surfaces 22 have been cleaned and/or to permit the user to suggest to the computer 14 that the cabin surfaces 22 should be purged once the user leaves the vehicle 12 (e.g., based on visual inspection by the respective user). In at least one example, a level of sterilization be selected via module 32; this is discussed more below as well.

FIG. 2 also illustrates telematics module 34. Module 34 may be any suitable telematics computing device configured to wirelessly communicate with other electronic devices—e.g., including a remote server (not shown) or even local devices such as a mobile device 70 (which may be carried by a user). Such wireless communication via telematics module 34 may include use of cellular technology (e.g., LTE, GSM, CDMA, and/or other cellular communication protocols), short range wireless communication technology (e.g., using Wi-Fi, Bluetooth, Bluetooth Low Energy (BLE), dedicated short range communication (DSRC), and/or other short range wireless communication protocols), or a combination thereof. Such communication includes so-called vehicle-to-vehicle (V2V) and vehicle-to-infrastructure (V2I) communications as well of which will be appreciated by those skilled in the art.

Mobile device 70 may be any suitable portable, electronic communication device. It may be used by a user to communicate with computer 14 via telematics module 34. For example, the user of the device 70 who desires to use vehicle 12 as an autonomous taxi may—via the device 70—request a ride, provide a desired destination, instruct the computer 14 to clean the surfaces 22 of the vehicle 12 before the user ingresses, and even to select a sterilization level of the surfaces therein. Non-limiting examples of mobile device 70 include a cellular telephone, a personal digital assistant (PDA), a Smart phone, a laptop or tablet computer having two-way communication capabilities (e.g., via a land and/or wireless connection), a netbook computer, and the like.

Mobile device 70 may communicate with telematics module 34 directly (e.g., via any suitable wireless peer-to-peer connection) or it may communicate with module 34 indirectly—e.g., via a wired and/or wireless communication network 72. This network 72 may comprise a land communication network, a wireless communication network, or a combination thereof, as will be appreciated by those skilled in the art. For example, a land communication network can enable connectivity to public switched telephone network (PSTN) such as that used to provide hardwired telephony, packet-switched data communications, internet infrastructure, and the like; further, the land communication network may facilitate V2I communication as well. A wireless communication network may include satellite communication architecture and/or may include cellular telephone communication over wide geographic region(s). Thus, in at least one example, network 72 includes any suitable cellular infrastructure that could include eNodeBs, serving gateways, base station transceivers, and the like. Further, network 72 may utilize any suitable existing or future cellular technology (e.g., including LTE, CDMA, GSM, etc.).

As will be discussed more below, one or more remote servers (not shown) may communicate with and/or instruct computer 14 via network 72. For example, in at least one example, the server may be associated with or owned by a vehicle manufacturer or other original equipment manufacturer. Via the communication, an authorized service technician may control computer 14, lighting system 16, etc. Further, from time-to-time, such remote servers may provide software updates or so-called software patches which may be installed in computer memory 42 (so that thereafter, processor 40 may execute updated programming instructions to clean the interior surfaces 22 of vehicle 12).

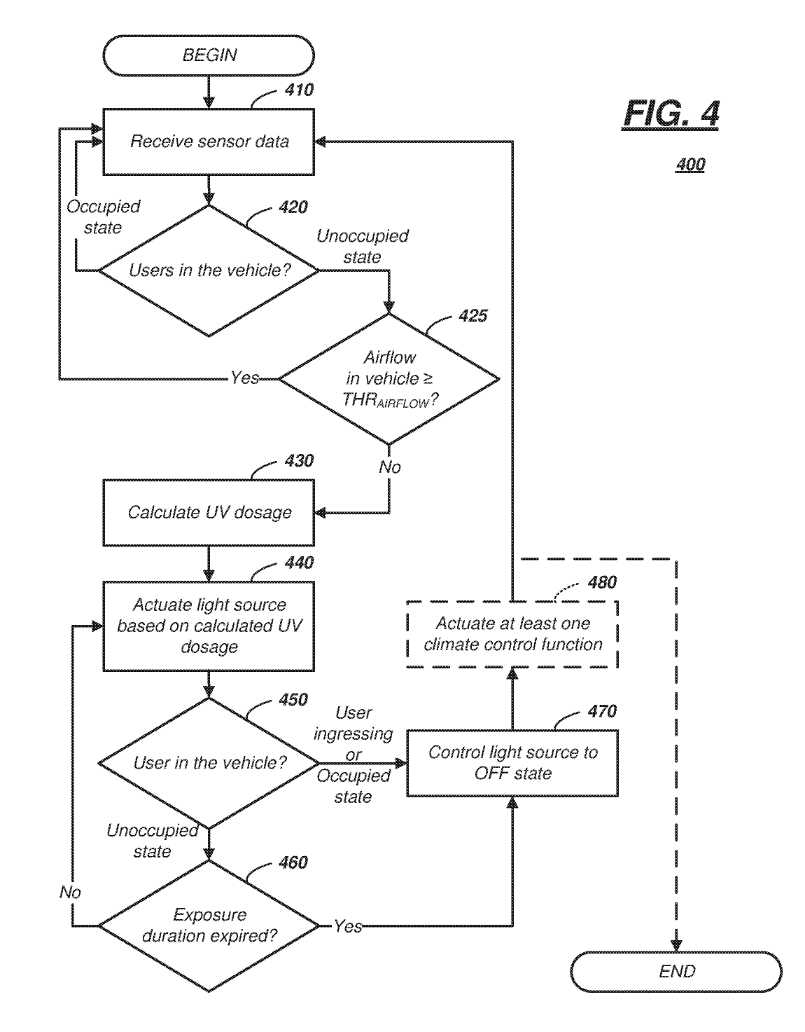

Turning now to FIG. 4, a process 400 is shown for cleaning the interior surfaces 22 of vehicle 12. The process begins with instructional block 410 which includes computer 14 receiving sensor data from one or more environmental sensors 18 in vehicle 12. Thus, for example, one or more cabin humidity sensors 18_{H} may receive data pertaining to a humidity measurement within vehicle cabin 20; one or more cabin temperature sensors 18_{T} may receive data pertaining to a temperature measurement within vehicle cabin 20; one or more surface moisture sensors 18_{SM} may receive data pertaining to a surface moisture measurement of a respective surface 22 within vehicle cabin 20; one or more UV sunlight sensors 18_{S} may receive data pertaining to a measurement of UV light upon a respective surface 22 within vehicle cabin 20; one or more airflow sensors 18_{A} may receive data pertaining to an airflow measurement within vehicle cabin 20; etc. When multiple sensor data is received from a same type of environmental sensor 18, computer 14 may average the values. Or in some instances, computer 14 may evaluate environmental conditions for one or more sub-regions 76 of cabin 20. The size and quantity of sub-regions 76 shown in FIG. 2 is merely an example (other quantities and/or sizes may be used). According to one example, computer 14 may determine to average multiple sensor data received regarding cabin humidity, cabin temperature, and cabin airflow; however, for an identified sub-region 76, it may determine an independent UV sunlight measurement, an independent surface moisture measurement, or the like. Other examples also exist. It should be appreciated that while block 410 is illustrated as a single block, in some examples, computer 14 may continue to receive sensor data throughout the process 400.

In block 420 which follows, computer 14 may determine whether any users are in the cabin 20 of vehicle 12. For example, computer 14 may receive an indication from occupant detection system 28—which may employ one or more sensing apparatuses discussed above and provide an electrical output received by processor 40 indicating an occupied state or an unoccupied state of vehicle 12. As discussed above, this determination may be based on motion sensing, proximity sensing, pressure sensing, infrared or thermal sensing, imaging sensing, or the like. When computer 14 determines an occupied state, the process loops back to block 410 and the computer 14 continues to receive additional environmental sensor data. And when computer 14 determines an unoccupied state, the process 400 proceeds to block 425.

In block 425, using sensor data from sensor(s) 18_{A}, the computer 14 may determine whether a relative airflow within the vehicle cabin 20 is greater than or equal to a threshold (THR_{AIRFLOW}). As used herein, a relative airflow is a movement of air within the cabin 20 of vehicle 12. According to one non-limiting example, threshold (THR_{AIRFLOW}) may be 1 meter per second (m/s); however, other examples exist. According to one example, the threshold (THR_{AIRFLOW}) corresponds to a value in which contaminants (e.g., the cells of bacteria or fungi, endospores, etc.) typically do not adhere and/or bind to one another—e.g., thus, mitigating a need for sterilization. Further, relative airflow greater than or equal to the threshold (THR_{AIRFLOW}) may suggest that a user is within or ingres sing the vehicle cabin 20, that a window of vehicle 12 is open, etc. In either instance, according to one example, no application of UV dosage occurs if sensed airflow is greater than or equal to threshold (THR_{AIRFLOW}). For example, when computer 14 determines that relative airflow THR_{AIRFLOW}, then the process may loop back to block 410, and the computer 14 may continue to receive additional environmental sensor data. And when computer 14 determines relative airflow<THR_{AIRFLOW}, the process 400 may proceed to block 430. (According to at least one example, process 400 loops back to block 410 at any time computer 14 determines (during block 440) that airflow within cabin 20 greater than or equal to threshold (THR_{AIRFLOW}). For example, if any light source 50 is actuated at the time of this determination, computer 14 immediately may change the source(s) 50 to a de-actuated state.)

In block 430, computer 14 may calculate a UV dosage for a light source 50 to be applied at a targeted interior surface 22 (e.g., which corresponds to an emission beam from respective source 50). The targeted interior surface 22 may comprise an entire object (e.g., such as an instrument panel, a center console, an arm rest, etc.)—or it may be for one or more sub-regions 76 of the cabin 20 (as discussed above). In some instances, block 430 may include determining a plurality of light emission parameters—e.g., such as a narrow emission band and/or center wavelength (λ), an emission intensity or flux (Φ), and an exposure duration (t_{EXPOS}). Each will be discussed in turn.

According to one example, computer 14 may to select one of a plurality of UV wavelengths (or center frequencies) as part of block 430. For example, different wavelengths could be determined by computer 14 by selectively actuating one or more light elements 56 of the light source 50 (e.g., wherein the elements 56 emit different UV wavelengths), by selectively actuating one or more of the filters 58 of the light source 50, or a combination thereof. Of course, in at least one example, the UV band and/or center frequency (λ) is predetermined and not selectable by computer 14.

As discussed above, block 430 comprises determining a UV dosage to be applied to the targeted interior surface 22—e.g., regardless of the UV band and center frequency (λ). As used herein, a dosage (also called fluence) is a quantity of UV light energy received at a unit area of interior surface 22 (e.g., micro-Joules per square centimeter or μJ/cm²). In general, dosage (D) can be expressed according to Equation 1.

D=Φ*t_{EXPOS}, where Φ (e.g., having units of micro-Watts per square centimeter or μW/cm²) is an intensity or radiant flux of UV light at surface 22 and t_{EXPOS} (having units of seconds (s)) is an exposure duration of the UV light. Equation 1:

As discussed above, computer 14 may calculate the dosage based on sensor data from one or more of the environmental sensors 18, as well as other factors (e.g., beam divergence, attenuation, etc.). More particularly, computer 14 may determine a dosage (D_{ENV}) based on environmental sensors data within the cabin 20, as illustrated in the non-limiting example shown as Equation 2. Stated differently, dosage (D) of Equation 1 may be modified as dosage (D_{ENV}), which also depends on environmental factors of cabin 20 such as temperature, humidity, surface moisture, UV sunlight, etc.

D_{ENV}=(Φ*t_{EXPOS})*r(h)*D_{T}*M)−D_{SUN}, where r(h) is a function of the relative humidity within cabin 20, where D_{T} is a function of temperature within cabin 20, where M is a function of moisture at the target surface 22, and where D_{SUN} is a correction factor based on the amount of ambient UV light (e.g., from the sun) which is impinging upon the targeted surface 22. Each will be explained in greater detail. Equation 2

The r(h) function may be defined accordingly: when sensor data from environmental sensor(s) 18_{H} indicates a relative humidity that is less than a threshold THR_{REL}_{_}_{HUM} (e.g., such as 60%), then r(h)=1; and when sensor data from sensor(s) 18_{H} indicates a relative humidity that is greater than or equal to threshold THR_{REL}_{_}_{HUM}, then 1<r(h)≤5. As used herein, relative humidity is a humidity within the air of the cabin 20 of vehicle 12. Thus, for example, the r(h) function may scale linearly, exponentially, or the like between 1 and 5 when threshold THR_{REL}_{_}_{HUM} is greater than 60% and less than 100%. Of course, the threshold THR_{REL}_{_}_{HUM} value of 60% and function values of 1<r(h)≤5 are merely examples; and other examples also exist.

The D_{T} function may be defined accordingly: when sensor data from environmental sensor(s) 18_{T} indicates a relative temperature that is less than or equal to a first temperature threshold THR_{TEMP}_{_}_{LOW} (e.g., such as 0° Celsius (° C.)), then D_{T}=[1+D_{T}_{_}_{LOW}(t)], where −1<D_{T}_{_}_{LOW}(t)<0; and when sensor data from environmental sensor(s) 18_{T} indicates a relative temperature that is greater than or equal to a second temperature threshold THR_{TEMP}_{_}_{HIGH} (e.g., such as 30° C.), then D_{T}=[1+D_{T}_{_}_{HIGH}(t)], where −1<D_{T}_{_}_{HIGH}(t)<0. As used herein, relative temperature is a temperature of the air within cabin 20 of vehicle 12. Thus, according to one example, when sensor 18_{T} indicates 0° C.≤relative temperature 30° C., then D_{T LOW}(t)=D_{T HIGH}(t)=0 and D_{T}=1.

When sensor 18_{T} indicates a relative temperature between 0° C. and a maximum low temperature (e.g., such as −20° C.), then D_{T}_{_}_{LOW}(t) is scaled between 0 and −1 (according to any suitable scaling). Similarly, when sensor 18_{T} indicates a relative temperature between 30° C. and a maximum high temperature (e.g., such as 50° C.), then D_{T}_{_}_{HIGH}(t) is scaled between 0 and −1 (according to any suitable scaling).

The M function may be defined accordingly: when sensor data from sensor(s) 18_{SM} indicates any moisture on the targeted interior surface 22, the value of M may be between a predetermined range (e.g., such as 2-10); and where no moisture exists on the targeted interior surface 22 (or when the moisture on surface 22 is less than a threshold THR_{MOIST}), then the value of M may be 1. The predetermined range (e.g., of 2-10) may be scaled in any suitable manner (e.g., linearly, exponentially, etc.), as already described above. Of course, these values also are merely examples, and other values may be used instead.

The value of D_{SUN} may be based on a measured value at the light detector 52 when the light source 50 is in the un-actuated state. For example, some surfaces 22 in cabin 20 may receive direct (or even reflected) sunlight comprising UV rays. Prior to actuation of the light source 50, detector 52 may measure this UV value and provide it to computer 14. Using this value, computer 14 may extrapolate the dosage attributable from the sunlight based on a surface area of target surface 22, and this extrapolated value may be assigned as D_{SUN}.

In accordance with Equation 2, it should be appreciated that computer 14 is not required to collect sensor data from each of a relative humidity sensor 18_{H}, a relative temperature sensor 18_{T}, a surface moisture sensor 18_{SM}, and a UV sunlight sensor 18_{S}. For example, where sensor data is not collected for relative humidity, r(h) may be assumed to equal one (e.g., 1). And for example, where sensor data is not collected for relative temperature, D_{T} may be assumed to equal one (e.g., 1). And for example, where sensor data is not collected for surface moisture, M may be assumed to equal one (e.g., 1). And for example, where sensor data is not collected for UV sunlight, D_{SUN} may be assumed to equal zero (e.g., 0).

Other factors may be used alternatively, or in addition to, those illustrated in Equation 2. For example, as discussed above, the computer 14, an authorized service technician, or even a user (e.g., via mobile device 70) may select a sterilization level. For example, the sterilization level may be based on a logarithmic kill ratio (e.g., how much of the contaminant is likely to be killed or destroyed); non-limiting examples include kill ratios of 90% (e.g., 90% of bacteria, viruses, fungi, etc. are killed), 99% (e.g., 99% of bacteria, viruses, fungi, etc. are killed), 99.99% (e.g., 99.99% of bacteria, viruses, fungi, etc. are killed), etc. Accordingly, dosage (D_{ENV}) may be modified based on the selected sterilization level—e.g., modified as dosage (D_{ENV-90}), dosage (D_{ENV-99}), dosage (D_{ENV-99.99}), etc. Accordingly, in many instances, dosage (D_{ENV-99.99}) may be larger than dosage (D_{ENV-90}) and dosage (D_{ENV-99}), and, in many instances, dosage (D_{ENV-99}) may be larger than dosage (D_{ENV-90}). The scaling factors may be based on testing or other empirical values. Tables I, II, and III below are merely illustrative examples illustrating UV intensity values, exposure durations (t_{EXPOS}), and dosages (D_{ENV-90}, D_{ENV-99}) for a few illustrative contaminants.

For purposes of illustration only, Table I below shows two different dosage examples based on different sterilization levels (e.g., in this particular instance, r(h)=D_{T}=M=1 and D_{SUN}=0).

| | UV | t_{EXPOS} [for | t_{EXPOS} [for | D_{ENV-90} | D_{ENV-99} |
|---|---|---|---|---|---|
| | Intensity | 90% kill | 99% kill | (μJ/ | (μJ/ |
| Contaminant | (μW/cm²) | ratio] (s) | ratio] (s) | cm²) | cm²) |
| BACTERIA | 1000 | 3 | 6.6 | 3000 | 6600 |
| ENDOSPORE | 1000 | 4.5 | 8.7 | 4500 | 8700 |
| FUNGI | 1000 | 44 | 88 | 44000 | 88000 |
| YEAST | 1000 | 3.3 | 6.6 | 3300 | 6600 |

For purposes of illustration only, Table II below shows an example of the higher relative humidity (e.g., in this particular instance, r(h)=5, D_{T}=M=1, and D_{SUN}=0).

| | | r(h) * | r(h) * | | |
|---|---|---|---|---|---|
| | | t_{EXPOS} | t_{EXPOS} | r(h) * | r(h) * |
| | UV | [for | [for | D_{ENV-90} | D_{ENV-99} |
| | Intensity | 90% kill | 99% kill | (μJ/ | (μJ/ |
| Contaminant | (μW/cm²) | ratio] (s) | ratio] (s) | cm²) | cm²) |
| BACTERIA | 1000 | 15 | 33 | 15000 | 33000 |
| ENDOSPORE | 1000 | 22.5 | 43.5 | 22500 | 43500 |
| FUNGI | 1000 | 220 | 440 | 220000 | 440000 |
| YEAST | 1000 | 16.5 | 33 | 16500 | 33000 |

For purposes of illustration only, Table III below shows an example of the relatively high surface moisture (e.g., in this particular instance, M=10, r(h)=D_{T}=1, and D_{SUN}=0).

| | | M * | M * | | |
|---|---|---|---|---|---|
| | | t_{EXPOS} | t_{EXPOS} | M * | M * |
| | UV | [for | [for | D_{ENV-90} | D_{ENV-90} |
| | Intensity | 90% kill | 99% kill | (μJ/ | (μJ/ |
| Contaminant | (μW/cm²) | ratio] (s) | ratio] (s) | cm²) | cm²) |
| BACTERIA | 1000 | 30 | 66 | 30000 | 66000 |
| ENDOSPORE | 1000 | 45 | 87 | 45000 | 87000 |
| FUNGI | 1000 | 440 | 880 | 440000 | 880000 |
| YEAST | 1000 | 33 | 66 | 33000 | 66000 |

In at least one example of block 430, sensor data from multiple environmental sensors 18 are used to determine the UV dosage. Further, in at least one example, computer 14—in its dosage calculation—uses sensor data from each of sensor 18_{T}, sensor 18_{H}, sensor 18_{S}, and sensor 18_{SM}.

While not required, computer 14 may identify a type of contaminant by determining a likelihood (e.g., a statistical probability) of the type of contaminant using measurement values of the environmental sensors 18, as well as other geographic data, calendar data, etc. For example, computer 14 may predict the presence of endospores and fungi by determining a relatively high humidity (e.g., about 90%), temperatures between 20-30° C., and a relatively high surface moisture (e.g., wherein M is between 8-10). Computer 14 via telematics module 34 may receive an indication of geographic location (e.g., Tampa, Fla., USA) and time of year (e.g., September), and based on this additional information, further may narrow a likely contaminant (e.g., to be a fungi or particular strain of fungi). In at least one example, the UV dosage is based on a contaminant type prediction using such a predictive algorithm; however, this is not required.

Returning to FIG. 4, following block 430, computer 14 may execute instructional block 440. In block 440, computer 14 controls and actuates a light source 50 based on UV dosage calculated in block 430. Thus, the light source 50 emits UV light according to the center wavelength (λ) and bandwidth and according to the calculated intensity (Φ). According to one non-limiting example, the intensity (Φ) may be between 500-2000 μW/cm². Due to beam divergence, attenuation, and other losses, computer 14 may emit UV light at an intensity (e.g., at the light source 50) that is greater than is expected to be received at the targeted surface 22 (and detector 52); these higher values may be determined empirically (at the vehicle manufacturer) or using the feedback loop discussed above. Further, according to at least one example, the total dosage (D_{ENV}) delivered to the respective surface 22 for any application period may be less than a maximum dosage (D_{MAX}) (e.g., one non-limiting example of maximum dosage (D_{MAX}) may be 1,000,000 μJ/cm² or 1 J/cm²), and this maximum dosage may be associated with minimizing interior component deterioration.

In block 440, computer 14 also may actuate a timer (e.g., via processor 40) to track the exposure time. This timer may be executed in software via processor 40 (or via an electrical timing circuit coupled to processor 40). Regardless, as discussed above, this UV light may be directed at the targeted interior surface 22 of cabin 20—which also may include a corresponding light detector 52. As the computer 14 may attempt to deliver UV light to the surface 22 according to the dosage, block 440 may include adjusting a magnitude of the emission intensity (Φ) based on the feedback data provided by detector 52 (as discussed above).

Following block 440, computer 14 may re-determine whether a user is in the vehicle 12 (block 450). This instruction may be similar or identical to block 420; therefore, it will not be re-described herein. Using this block, computer 14 may repeatedly check the cabin 20 for a user ingressing vehicle 12 (e.g., during the transmission of UV light). In at least some examples, block 450 may include determining that a user is approaching vehicle 12, a user is opening a vehicle door, or the like. When computer 14 determines in block 450 that vehicle 12 remains in the unoccupied state, then process 400 may proceed to block 460. And when computer 14 determines that the vehicle is in an occupied state or about to be occupied, then process 400 proceeds to block 470 (discussed below).

In block 460, computer 14 determines whether the exposure duration (t_{EXPOS}) has expired by comparing the current run time of the timer (previously initiated) with the exposure duration (t_{EXPOS}). When computer 14 determines that vehicle 12 the exposure duration (t_{EXPOS}) has not expired, then process 400 may loop back to block 440. And when computer 14 determines that the exposure duration (t_{EXPOS}) has expired, then process 400 proceeds to block 470. Thus, computer 14 may execute blocks 440, 450, and 460 repeatedly—e.g., checking the occupancy state of the vehicle 12 and whether the exposure duration (t_{EXPOS}) has expired. In the latter case (as discussed above), de-actuating the light source 50 at the expiration of the exposure duration (t_{EXPOS}) inhibits overdosing of targeted surface 22—e.g., and mitigating premature deterioration of the vehicle interior components.

In block 470, computer 14 actuates the light source 50 to an OFF state. As discussed above, this may occur following a user ingress attempt or a user ingressing the vehicle 12 (e.g., block 450) or following the expiration of an exposure duration (e.g., block 460). During instances when multiple light sources 50 are dosing one or more interior surfaces 22, and when a user ingress is detected (block 450), computer 14 may de-actuate all light sources 50 via instructional block 470.

Following block 470, process 400 may proceed to block 480, to block 410, or end. For example, optional instructional block 480 may comprise computer 14 actuating at least one climate control parameter based on the sensor data collected in block 410 and/or based on the calculated UV dosage of block 430. As used herein, actuating a climate control parameter is a computer-actuated operation of the climate control system 30: that, when the parameter is adjusted, changes a temperature of the vehicle cabin 20; that, when the parameter is adjusted, changes a humidity within the cabin 20; and/or that, when the parameter is adjusted, changes a volume of forced air into the cabin 20. Non-limiting examples include increasing the temperature of the cabin 20, decreasing the humidity within the cabin 20, and increasing the amount of forced air (e.g., through HVAC vents into the cabin 20).

According to at least one example, one or more climate control parameters are actuated by computer 14 in response to a determined contamination type. In this manner, when the light source(s) 50 are in the OFF state, additional contaminant growth is minimized.

Following block 480, the process 400 either ends or loops back and repeats (at least a portion thereof) beginning with block 410. Similarly, when optional block 480 is omitted, the process proceeds from block 470 to either block 410 or simply ends.

Other examples exist. For example, block 470 may be executed when computer 14 determines that light source 50 is actuated but light detector 52 is receiving less than a threshold (e.g., less than 100 μJ/cm²) amount of UV light—e.g., indicating a misalignment of source 50 and detector 52. As a result, computer 14 may execute a shut-off instruction and may generate a diagnostic trouble code and not actuate the light source 50 until the vehicle 12 has been serviced by an authorized service technician.

Similarly, according to at least one example, block 470 may be executed when computer 14 determines that light source 50 is actuated but light detector 52 is receiving UV light within a threshold range (e.g., 100-500 μJ/cm²)—e.g., indicating a potential fault at source 50 and/or detector 52. Similarly, computer 14 may execute a shut-off instruction and may generate a diagnostic trouble code and not actuate the light source 50 until the vehicle 12 has been serviced by an authorized service technician.

Similarly, according to at least one example, block 470 may be executed when computer 14 determines that light source 50 is actuated but light detector 52 is receiving UV light greater than a threshold (e.g., 2000 μJ/cm²)—e.g., indicating a potential fault at source 50. Similarly, computer 14 may execute a shut-off instruction and may generate a diagnostic trouble code and not actuate the light source 50 until the vehicle 12 has been serviced by an authorized service technician.

According to at least one example, in block 420, computer 14 also may determine window state data and/or controls the state of the vehicle windows (e.g., from an open state to a closed state)—e.g., prior to actuating the light source 50. In this manner, UV light from the source 50 may be contained within the vehicle 12—e.g., particularly if the windows are manufactured with a protective UV-blocking film, polarizing glazing, or the like.

Tables I, II, and III illustrated a constant intensity value (e.g., 1000 μW/cm²) of UV light received at the surface 22, and the exposure duration (t_{EXPOS}) was increased in some instances (e.g., by a multiplier) to vary UV dosage. According to another example, UV dosage may be varied by changing the UV intensity (Φ), changing the exposure duration (t_{EXPOS}), or a combination thereof. Of course, when computer 14 increases the UV intensity, the intensity still may be bounded by a threshold range or maximum (e.g., between 500-2000 μW/cm²).

Thus, there has been described a cleaning system for a vehicle. The system includes a computer and a lighting system. The computer is programmed to receive sensor data from one or more environmental sensors in the vehicle cabin, and then, based on the data, determine a light dosage to emit from the lighting system toward an interior surface of the cabin.

In general, the computing systems and/or devices described may employ any of a number of computer operating systems, including, but by no means limited to, versions and/or varieties of the Ford SYNC® application, AppLink/Smart Device Link middleware, the Microsoft® Automotive operating system, the Microsoft Windows® operating system, the Unix operating system (e.g., the Solaris® operating system distributed by Oracle Corporation of Redwood Shores, Calif.), the AIX UNIX operating system distributed by International Business Machines of Armonk, N.Y., the Linux operating system, the Mac OSX and iOS operating systems distributed by Apple Inc. of Cupertino, California, the BlackBerry OS distributed by Blackberry, Ltd. of Waterloo, Canada, and the Android operating system developed by Google, Inc. and the Open Handset Alliance, or the QNX® CAR Platform for Infotainment offered by QNX Software Systems. Examples of computing devices include, without limitation, an on-board vehicle computer, a computer workstation, a server, a desktop, notebook, laptop, or handheld computer, or some other computing system and/or device.

Computing devices generally include computer-executable instructions, where the instructions may be executable by one or more computing devices such as those listed above. Computer-executable instructions may be compiled or interpreted from computer programs created using a variety of programming languages and/or technologies, including, without limitation, and either alone or in combination, Java™, C, C++, Visual Basic, Java Script, Perl, etc. Some of these applications may be compiled and executed on a virtual machine, such as the Java Virtual Machine, the Dalvik virtual machine, or the like. In general, a processor (e.g., a microprocessor) receives instructions, e.g., from a memory, a computer-readable medium, etc., and executes these instructions, thereby performing one or more processes, including one or more of the processes described herein. Such instructions and other data may be stored and transmitted using a variety of computer-readable media.

A computer-readable medium (also referred to as a processor-readable medium) includes any non-transitory (e.g., tangible) medium that participates in providing data (e.g., instructions) that may be read by a computer (e.g., by a processor of a computer). Such a medium may take many forms, including, but not limited to, non-volatile media and volatile media. Non-volatile media may include, for example, optical or magnetic disks and other persistent memory. Volatile media may include, for example, dynamic random access memory (DRAM), which typically constitutes a main memory. Such instructions may be transmitted by one or more transmission media, including coaxial cables, copper wire and fiber optics, including the wires that comprise a system bus coupled to a processor of a computer. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, or any other medium from which a computer can read.

Databases, data repositories or other data stores described herein may include various kinds of mechanisms for storing, accessing, and retrieving various kinds of data, including a hierarchical database, a set of files in a file system, an application database in a proprietary format, a relational database management system (RDBMS), etc. Each such data store is generally included within a computing device employing a computer operating system such as one of those mentioned above, and are accessed via a network in any one or more of a variety of manners. A file system may be accessible from a computer operating system, and may include files stored in various formats. An RDBMS generally employs the Structured Query Language (SQL) in addition to a language for creating, storing, editing, and executing stored procedures, such as the PL/SQL language mentioned above.

In some examples, system elements may be implemented as computer-readable instructions (e.g., software) on one or more computing devices (e.g., servers, personal computers, etc.), stored on computer readable media associated therewith (e.g., disks, memories, etc.). A computer program product may comprise such instructions stored on computer readable media for carrying out the functions described herein.

The processor is implemented via circuits, chips, or other electronic component and may include one or more microcontrollers, one or more field programmable gate arrays (FPGAs), one or more application specific circuits ASICs), one or more digital signal processors (DSPs), one or more customer integrated circuits, etc. The processor may be programmed to process the sensor data. Processing the data may include processing the video feed or other data stream captured by the sensors to determine the roadway lane of the host vehicle and the presence of any target vehicles. As described below, the processor instructs vehicle components to actuate in accordance with the sensor data. The processor may be incorporated into a controller, e.g., an autonomous mode controller.

The memory (or data storage device) is implemented via circuits, chips or other electronic components and can include one or more of read only memory (ROM), random access memory (RAM), flash memory, electrically programmable memory (EPROM), electrically programmable and erasable memory (EEPROM), embedded MultiMediaCard (eMMC), a hard drive, or any volatile or non-volatile media etc. The memory may store data collected from sensors.

The disclosure has been described in an illustrative manner, and it is to be understood that the terminology which has been used is intended to be in the nature of words of description rather than of limitation. Many modifications and variations of the present disclosure are possible in light of the above teachings, and the disclosure may be practiced otherwise than as specifically described.

1. A method, comprising:
at a vehicle computer:
receiving data from at least one environmental sensor;
based on the data, determining an ultraviolet (UV) dosage for an interior surface of a cabin; and
based on the determination, controlling UV light according to the dosage.

2. The method of claim 1, wherein controlling further comprises actuating a light source within the cabin which directs the light toward the surface.

3. The method of claim 1, wherein the light comprises a band within 240-280 nanometers.

4. The method of claim 1, wherein the dosage is based on a light intensity, an exposure duration, and at least one function based on the data.

5. The method of claim 4, further comprising de-actuating a light source at an expiration of the duration.

6. The method of claim 1, further comprising adjusting at least one climate control parameter based on the determination.

7. The method of claim 1, wherein controlling further comprises changing an intensity of the light based on feedback from a detector at the surface.

8. The method of claim 1, wherein the determination further comprises increasing an intensity of the light based on a relative humidity being greater than a threshold.

9. The method of claim 1, wherein the determination further comprises decreasing an intensity of the light based on a relative temperature being less than a first threshold or greater than a second threshold.

10. The method of claim 1, wherein the determination further comprises increasing an intensity of the light based on moisture at the surface being greater than a threshold.

11. The method of claim 1, wherein the determination further comprises decreasing an intensity of the light based on a measurement of UV sunlight at the surface.

12. The method of claim 1, further comprising inhibiting UV light emission based on an occupied state of the vehicle or a user ingres sing.

13. The method of claim 1, further comprising inhibiting UV light emission based on relative airflow being greater than a threshold.

14. The method of claim 1, further comprising inhibiting UV light emission based an open state of vehicle windows.

15. The method of claim 1, wherein the dosage is based on a selected sterilization level.

16. A system, comprising:
a computer, comprising processor and memory storing instructions executable by the processor, the instructions comprising, to:
receive data from at least one environmental sensor;
based on the data, determine an ultraviolet (UV) dosage for an interior surface of a cabin; and
based on the determination, control UV light according to the dosage.

17. The system of claim 16, further comprising a lighting system coupled to the computer.

18. The system of claim 17, wherein the lighting system comprises a light source and a detector which provides UV light intensity feedback.

19. The system of claim 15, wherein the instructions further comprise, to: determine the dosage based on one of a relative humidity, a relative temperature, or a moisture at the surface.

20. The system of claim 15, wherein the instructions further comprise, to: adjust the dosage based on a measurement of UV sunlight at the surface.
